(19) United States
(12) Patent Application Publication
(10) Pub. No.: US 2015/0342864 A1
(43) Pub. Date: Dec. 3, 2015

(54) COMPOSITION COMPRISING A SUPERABSORBENT POLYMER AND AN ORGANOPOLYSILOXANE ELASTOMER

(71) Applicant: L'OREAL, Paris (FR)

(72) Inventor: Frédérique Labatut, Paris (FR)

(21) Appl. No.: 14/654,135

(22) PCT Filed: Dec. 20, 2013
(86) PCT No.: PCT/EP2013/077607

§ 371 Date: Jun. 19, 2015

Related U.S. Application Data
(60) Provisional application No. 61/751,976, filed on Jan. 14, 2013.

(30) Foreign Application Priority Data
Dec. 21, 2012 (FR) 1262577

(51) Int. Cl. A61K 8/89; A61Q 19/00; A61Q 5/00; A61K 8/06; A61K 8/81

(52) U.S. Cl. CPC A61K 8/89; A61K 8/062; A61K 8/064; A61K 8/8152; A61Q 5/00; A61Q 19/00; A61K 2800/594

(57) ABSTRACT

The subject of the present invention is a composition for topical application in the form of an oil-in-water or water-in-oil emulsion comprising at least one superabsorbent polymer and at least one organopolysiloxane elastomer. The subject of the invention is also the use of this composition in the cosmetics or dermatology field, and in particular for caring for, protecting and/or making up bodily or facial skin, or for haircare. The present invention makes it possible to obtain a composition which is soft on application, while at the same time providing comfort and moisturization, and stable over time. In particular, the present invention makes it possible to obtain an opalescent translucent gel which has a specific cosmeticity: stringy when taken up, with a significant “cushion” effect when massaging is begun (consistency and thickness under the finger on application) or even slightly dragging until the texture breaks under the fingers while at the same time keeping an application which ends with a certain glide.

The present invention relates to a composition for topical application in the form of an oil-in-water or water-in-oil emulsion comprising at least one superabsorbent polymer and at least one organopolysiloxane elastomer, and to the use of said composition in the cosmetics and dermatology fields, and in particular for caring for, protecting and/or making up bodily or facial skin, or for haircare.

For various reasons associated in particular with greater comfort of use (softness, emollience and the like), current cosmetic compositions are usually in the form of an emulsion of the oil-in-water (O/W) type consisting of an aqueous dispersing continuous phase and an oily dispersed discontinuous phase, or of an emulsion of the water-in-oil (W/O) type consisting of an oily dispersing continuous phase and an aqueous dispersed discontinuous phase.

In order to improve the feel properties of these emulsions, silicone elastomers may be used; these compounds provide softness and better comfort on application, but can be difficult to stabilize in the emulsion, thereby limiting their incorporation into this type of composition, in particular at high contents. Moreover, when they are used at a high content, silicone elastomers can cause an unwanted oily/greasy effect on the skin.

Furthermore, the chemical nature of the silicone elastomer imposes limits with regard to the nature of the oils making up the oil phase: for reasons of feasibility and stability, it must contain predominantly silicone oils and/or volatile oils which do not provide sufficient care effects on the skin, such as nutrition of the skin, and effects that are visible and felt, in particular because the oil phase comprises essentially volatile oils which do not remain on the skin and cannot nourish the skin.

There remains therefore the need for a composition which does not have the drawbacks of the prior art and in particular which is soft on application, while at the same time providing comfort and moisturization. In particular, there remains the need for a composition which exhibits novel sensoriality while at the same time limiting the risks of destabilization of the formula.

The applicant has discovered that the addition of at least one superabsorbent polymer to a composition comprising at least one silicone elastomer makes it possible to limit the oily/greasy effect that the silicone elastomer introduces, while at the same time obtaining good stability of the composition, even at a high silicone elastomer content.

Thus, a subject of the present invention is a composition for topical application in the form of an oil-in-water or water-in-oil emulsion comprising at least one superabsorbent polymer and at least one organopolysiloxane elastomer.

The present invention makes it possible to obtain a composition which has a specific cosmeticity: stringy when taken up, with a significant “cushion effect” when massaging is begun (consistency and thickness under the finger on application) or even slightly dragging until the texture breaks under the fingers while at the same time keeping an application which ends with a certain glide.

Moreover, the addition of a small amount of superabsorbent polymer makes it possible to incorporate a greater amount of silicone elastomer into the composition without destabilizing it, and also enables the use of a smaller amount of silicone elastomer for the same technical effect observed.

A subject of the invention is also the use of said composition in the cosmetics or dermatology field, and in particular for caring for, protecting and/or making up bodily or facial skin, or for haircare.

In that which follows, the expression “at least one” is equivalent to “one or more” and, unless otherwise indicated, the limits of a range of values are included within this range.

### Superabsorbent Polymers

The term “superabsorbent polymer” is intended to mean a polymer that is capable in its dry form of spontaneously absorbing at least 20 times its own weight of aqueous fluid, in particular of water and in particular distilled water. Such superabsorbent polymers are described in the publication “Absorbent polymer technology, Studies in polymer science 8” by L. Brannon-Pappas and R. Harland, published by Elsevier, 1990.

These polymers have a large capacity for absorbing and retaining water and aqueous fluids. After absorption of the aqueous liquid, the polymer particles thus engorged with aqueous fluid remain insoluble in the aqueous fluid and thus conserve their individualized particulate state.

The superabsorbent polymer may have a water-absorbing capacity ranging from 20 to 2000 times its own weight (i.e. 20 g to 2000 g of absorbed water per gram of absorbent polymer), preferably from 30 to 1500 times and better still from 50 to 1000 times. These water absorption characteristics are defined under normal temperature (25° C.) and pressure (760 mmHg, i.e. 100000 Pa) conditions and for distilled water.

The value of the water-absorbing capacity of a polymer may be determined by dispersing 0.5 g of polymer(s) in 150 g of a water solution, waiting for 20 minutes, filtering the unabsorbed solution through a 150 μm filter for 20 minutes and weighing the unabsorbed water.

The superabsorbent polymer used in the composition of the invention is in the form of particles. Preferably, the superabsorbent polymer has, in the dry or nonhydrated state, an average size of less than or equal to 100 μm, preferably less than or equal to 50 μm, ranging for example from 10 to 100 μm, preferably from 15 to 50 μm, and better still from 20 to 30 μm.

The average size of the particles corresponds to the weight-average diameter (D₅₀) measured by laser particle size analysis or another equivalent method known to those skilled in the art.

Once hydrated, these particles swell, forming soft particles having an average size that can range from 10 μm to 1000 μm, preferentially from 20 μm to 500 μm and even more preferentially from 50 μm to 400 μm.

Preferably, the superabsorbent polymers used in the present invention are in the form of spherical particles.

Mention may be made in particular of absorbent polymers chosen from:
crosslinked sodium polyacrylates, for instance those sold under the names Octacare X100, X110 and RM100 by the company Innospec Active Chemicals, those sold under the names Flocare GB300 and Flosorb 500 by the company SNF, those sold under the names Luquasorb 1003, Luquasorb 1010, Luquasorb 1280 and Luquasorb 1110 by the company BASF, those sold under the names Water Lock G400 and G430 (INCI name: Acrylamide/Sodium acrylate copolymer) by the company Grain Processing, or else Aquakeep® 10 SH NF proposed by the company Sumitomo Seika,
starches grafted with an acrylic polymer (homopolymer or copolymer) and in particular with sodium polyacrylate, such as those sold under the name Sanfresh ST-100MC by the company Sanyo Chemical Industries or Makimousse 25 or Makimousse 12 by the company Daito Kasei (INCI name: Sodium polyacrylate starch),
hydrolysed starches grafted with an acrylic polymer (homopolymer or copolymer) and in particular acryloacrylamide/sodium acrylate copolymer, such as those sold under the names Water Lock A-240, A-180, B-204, D-223, A-100, C-200 and D-223 by the company Grain Processing (INCI name: Starch/acrylamide/sodium acrylate copolymer),
polymers based on starch, on gum and on cellulose derivative, such as the product containing starch, guar gum and sodium carboxymethylcellulose, sold under the name Lysorb 220 by the company Lysac,
and mixtures thereof.

The superabsorbent polymers used in the present invention may be crosslinked or noncrosslinked. They are preferably chosen from crosslinked polymers.

The superabsorbent polymers used in the present invention are preferably crosslinked acrylic homopolymers or copolymers, which are preferably neutralized, and which are in particulate form.

Preferably, the superabsorbent polymer is chosen from crosslinked sodium polyacrylates, preferably in the form of particles with an average size (or average diameter) of less than or equal to 100 microns, more preferably in the form of spherical particles. These polymers preferably have a capacity for absorbing water containing 0.9% of NaCl of from 10 to 100 g/g, preferably from 20 to 80 g/g and better still from 30 to 80 g/g.

The superabsorbent polymer(s) may be present in the composition of the invention in an active material content ranging, for example, from 0.01% to 5% by weight, preferably from 0.01% to 1% by weight or even from 0.02% to 0.7% by weight relative to the total weight of the composition.

### Organopolvsiloxane Elastomer

The composition of the invention contains at least one organopolysiloxane elastomer, also called “silicone elastomer” in the rest of the description, which may be noncrosslinked or at least partially crosslinked. The term “elastomer” is intended to mean a deformable, flexible, solid material having viscoelastic properties and in particular the consistency of a sponge or of a flexible sphere. Its modulus of elasticity is such that this material withstands deformation and has a limited ability to extend and to contract. This material is capable of regaining its original shape after stretching. This elastomer is formed from high-molecular-weight polymer chains, the mobility of which is limited by a uniform network of crosslinking points.

According to one particular embodiment of the invention, the organopolysiloxane elastomer(s) used in the composition is (are) partially or totally crosslinked. They may then be in the form of particles. These particles can have any shape and can, for example, be spherical, flat or amorphous.

When they are included in an oil phase, these organopolysiloxane elastomers transform, according to the level of oil phase used, into a product with a spongy appearance when they are used in the presence of low contents in the oil phase, or into a homogeneous gel in the presence of higher quantities of oil phase. The oil phase can be completely or partially gelled by these elastomers.

Thus, the elastomers of the invention may be conveyed in the form of an anhydrous gel constituted of an organopolysiloxane elastomer and an oil phase. The oil phase used while manufacturing the anhydrous organopolysiloxane elastomer gel contains one or more oils that are liquid at ambient temperature (25° C.) chosen from hydrocarbon-based oils and/or silicone oils. Advantageously, the oil phase is a silicone-based liquid phase, containing one or more oils chosen from polydimethylsiloxanes with straight or cyclic chains, which are liquid at ambient temperature, optionally containing an alkyl or aryl chain that is pendent or at the chain end, the alkyl chain having from 1 to 6 carbon atoms.

The organopolysiloxane elastomers used according to the invention may be chosen from crosslinked polymers described in application EP-A-0295886 and from those described in patent U.S. Pat. No. 5,266,321. They may be emulsifying or non-emulsifying. The term “emulsifying organopolysiloxane elastomers” is intended to mean a silicone elastomer comprising at least one hydrophilic chain, it being possible for this chain to be in particular oxyalkylenated or glycerolated.

According to a specific embodiment of the invention, the emulsifying silicone elastomer(s) can be chosen from polyoxyalkylenated silicone elastomers, polyglycerolated silicone elastomers and mixtures thereof.

### Polyoxyalkylenated Silicone Elastomers

The polyoxyalkylenated silicone elastomer is a crosslinked organopolysiloxane which can be obtained via a crosslinking addition reaction of a diorganopolysiloxane containing at least one hydrogen bonded to silicon and of a polyoxyalkylene containing at least two ethylenically unsaturated groups.

Preferably, the polyoxyalkylenated crosslinked organopolysiloxane is obtained via a crosslinking addition reaction (A1) of a diorganopolysiloxane containing at least two hydrogens each bonded to a silicon and (B1) of a polyoxyalkylene containing at least two ethylenically unsaturated groups, in particular in the presence (C1) of a platinum catalyst, such as, for example, described in the patents U.S. Pat. No. 5,236,986 and U.S. Pat. No. 5,412,004.

In particular, the organopolysiloxane can be obtained via a reaction of a dimethylvinylsiloxy-terminated polyoxyalkylene (in particular a polyoxyethylene and/or polyoxypropylene) and of a trimethylsiloxy-terminated methylhydropolysiloxane, in the presence of a platinum catalyst.

The organic groups bonded to the silicon atoms of the compound (A1) can be alkyl groups having from 1 to 18 carbon atoms, such as methyl, ethyl, propyl, butyl, octyl, decyl, dodecyl (or lauryl), myristyl, cetyl or stearyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon-based groups, such as an epoxy group, a carboxylate ester group or a mercapto group.

The compound (A1) can thus be chosen from methylhydropolysiloxanes trimethylsiloxy-terminated, dimethylsiloxane/methylhydrosiloxane copolymers trimethylsiloxy-terminated, cyclic dimethylsiloxane/methylhydrosiloxane copolymers, or dimethylsiloxane/methylhydrosiloxane/laurylmethylsiloxane copolymers trimethylsiloxy-terminated.

Compound (C1) is the catalyst for the crosslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

Advantageously, the polyoxyalkylenated silicone elastomers can be formed from divinyl compounds, in particular polyoxyalkylenes having at least two vinyl groups, which react with Si—H bonds of a polysiloxane.

The polyoxyalkylenated silicone elastomer according to the invention is preferably mixed with at least one hydrocarbon-based oil and/or one silicone oil in order to form a gel. In these gels, the polyoxyalkylenated elastomer can be in the form of non-spherical particles.

Polyoxyalkylenated elastomers are described in particular in the patents U.S. Pat. No. 5,236,986, U.S. Pat. No. 5,412,004, U.S. Pat. No. 5,837,793 and U.S. Pat. No. 5,811,487.

Use may be made, as polyoxyalkylenated silicone elastomer, of those sold under the names KSG-21, KSG-20, KSG-30, KSG-31, KSG-32, KSG-33, KSG-210, KSG-310, KSG-320, KSG-330, KSG-340, X-226146, KSG-380Z and KSG-320Z by the company Shin-Etsu, and DC9010 and DC9011 by the company Dow Corning.

According to a preferred embodiment, use will be made of the polyoxyalkylenated silicone elastomer sold under the name KSG-210 by the company Shin-Etsu.

### Polyglycerolated Silicone Elastomers

The polyglycerolated silicone elastomer is a crosslinked organopolysiloxane elastomer which can be obtained via a crosslinking addition reaction of a diorganopolysiloxane containing at least one hydrogen bonded to silicon and of polyglycerolated compounds containing ethylenically unsaturated groups, in particular in the presence of a platinum catalyst.

Preferably, the crosslinked organopolysiloxane elastomer is obtained via a crosslinking addition reaction (A2) of diorganopolysiloxane containing at least two hydrogens each bonded to a silicon, and (B2) of glycerolated compounds containing at least two ethylenically unsaturated groups, in particular in the presence (C2) of a platinum catalyst.

In particular, the organopolysiloxane can be obtained via a reaction of a polyglycerolated compound dimethylvinylsiloxy-terminated and of a methylhydropolysiloxane trimethylsiloxy-terminated, in the presence of a platinum catalyst.

Compound (A2) is the base reagent for the formation of an organopolysiloxane elastomer and the crosslinking takes place via an addition reaction of compound (A2) with compound (B2) in the presence of the catalyst (C2).

Compound (A2) is in particular an organopolysiloxane containing at least 2 hydrogen atoms bonded to different silicon atoms in each molecule.

Compound (A2) may have any molecular structure, in particular a linear-chain or branched-chain structure or a cyclic structure.

Compound (A2) may have a viscosity at 25° C. ranging from 1 to 50 000 centistokes, in particular so as to be miscible with compound (B2).

The organic groups bonded to the silicon atoms in compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from the methyl, phenyl and lauryl groups.

Compound (A2) may thus be chosen from methylhydropolysiloxanes trimethylsiloxy-terminated, dimethylsiloxane/methylhydrosiloxane copolymers trimethylsiloxy-terminated, dimethylsiloxane/methylhydrosiloxane cyclic copolymers and dimethylsiloxane/methylhydrosiloxane/laurylmethylsiloxane copolymers trimethylsiloxy-terminated.

Compound (B2) may be a polyglycerolated compound corresponding to formula (B2′) below:

CₘH₂ₘ₋₁—O−[Gly]ₙ−CₘH₂ₘ₋₁ (B2′)

in which m is an integer ranging from 2 to 6, n is an integer ranging from 2 to 200, preferably ranging from 2 to 100, preferably ranging from 2 to 50, preferably n ranging from 2 to 20, preferably ranging from 2 to 10 and preferentially ranging from 2 to 5, and in particular equal to 3; Gly denotes:

—CH₂—CH(OH)—CH₂—O— or —CH₂—CH(CH₂OH)—O—

Advantageously, the sum of the number of ethylenic groups per molecule of compound (B2) and of the number of hydrogen atoms bonded to silicon atoms per molecule of compound (A2) is at least 4.

It is advantageous for compound (A2) to be added in an amount such that the molecular ratio between the total amount of hydrogen atoms bonded to silicon atoms in compound (A2) and the total amount of all the ethylenically unsaturated groups in compound (B2) is within the range from 1/1 to 20/1.

Compound (C2) is the catalyst for the crosslinking reaction, and is in particular chloroplatinic acid, chloroplatinic acid/olefin complexes, chloroplatinic acid/alkenylsiloxane complexes, chloroplatinic acid/diketone complexes, platinum black and platinum-on-support.

The catalyst (C2) is preferably added in an amount of from 0.1 to 1000 parts by weight and better still from 1 to 100 parts by weight, as platinum metal proper, per 1000 parts by weight of the total amount of compounds (A2) and (B2).

The polyglycerolated silicone elastomer according to the invention is generally mixed with at least one hydrocarbon-based oil and/or one silicone oil in order to form a gel. In these gels, the polyglycerolated elastomer is often in the form of non-spherical particles.

Such elastomers are described in particular in patent application WO 2004/024798.

Use may be made, as polyglycerolated silicone elastomers, of those sold under the names KSG-710, KSG-810, KSG-820, KSG-830, KSG-840 and KSG-820Z by the company Shin-Etsu.

The term “non-emulsifying organopolysiloxane elastomers” is intended to mean organopolysiloxane elastomers that do not contain a hydrophilic chain, such as polyoxyalkylene or polyglycerol units.

According to one particular embodiment of the invention, the non-emulsifying silicone elastomer(s) is (are) a crosslinked organopolysiloxane elastomer or elastomers which can be obtained via a crosslinking addition reaction of a diorganopolysiloxane containing at least one hydrogen bonded to silicon and of a diorganopolysiloxane containing ethylenically unsaturated groups bonded to silicon, in particular in the presence of a platinum catalyst; or via a dehydrogenation crosslinking condensation reaction between a hydroxyl-terminated diorganopolysiloxane and a diorganopolysiloxane containing at least one hydrogen bonded to silicon, in particular in the presence of an organotin compound; or via a crosslinking condensation reaction of a hydroxyl-terminated diorganopolysiloxane and of a hydrolysable organopolysilane; or via thermal crosslinking of an organopolysiloxane, in particular in the presence of an organoperoxide catalyst; or via crosslinking of an organopolysiloxane by high-energy radiation such as gamma rays, ultraviolet rays or an electron beam.

Preferably, the crosslinked organopolysiloxane elastomer is obtained via a crosslinking addition reaction (A) of a diorganopolysiloxane containing at least two hydrogen atoms each bonded to a different silicon atom, and (B) of a diorganopolysiloxane containing at least two ethylenically unsaturated groups bonded to silicon, in particular in the presence (C) of a platinum catalyst, for instance as described in application EP-A-295 886.

In particular, the organopolysiloxane may be obtained via a reaction of a dimethylvinylsiloxy-terminated dimethylpolysiloxane and of a trimethylsiloxy-terminated methylhydropolysiloxane, in the presence of a platinum catalyst.

Compound (A) is the base reagent for the formation of organopolysiloxane elastomer, and the crosslinking is performed by addition reaction of compound (A) with compound (B) in the presence of the catalyst (C).

Compound (A) is advantageously a diorganopolysiloxane containing at least two lower (for example of C₂-C₄) alkenyl groups; the lower alkenyl group may be chosen from vinyl, allyl and propenyl groups. These lower alkenyl groups can be located at any position on the organopolysiloxane molecule but are preferably located at the ends of the organopolysiloxane molecule. The organopolysiloxane (A) may have a branched-chain, linear-chain, cyclic or network structure, but the linear-chain structure is preferred.

Compound (A) may have a viscosity ranging from the liquid state to the gum state.

Preferably, compound (A) has a viscosity of at least 100 centistokes at 25° C.

The organopolysiloxanes (A) may be chosen from methylvinylsiloxanes, methylvinylsiloxane/dimethylsiloxane copolymers, dimethylvinylsiloxy-terminated dimethylpolysiloxanes, dimethylvinylsiloxy-terminated dimethylsiloxane/methylphenylsiloxane copolymers, dimethylvinylsiloxy-terminated dimethylsiloxane/diphenylsiloxane/methylvinylsiloxane copolymers, trimethylsiloxy-terminated dimethylsiloxane/methylvinylsiloxane copolymers, trimethylsiloxy-terminated dimethylsiloxane/methylphenylsiloxane/methylvinylsiloxane copolymers, dimethylvinylsiloxy-terminated methyl(3,3,3-trifluoropropyl)polysiloxanes, and dimethylvinylsiloxy-terminated dimethylsiloxane/methyl(3,3,3-trifluoropropyl)siloxane copolymers.

Compound (B) is in particular an organopolysiloxane containing at least 2 hydrogens bonded to silicon in each molecule and is thus the crosslinking agent for compound (A).

Advantageously, the sum of the number of ethylenic groups per molecule of compound (A) and the number of hydrogen atoms bonded to silicon per molecule of compound (B) is at least 4.

Compound (B) may be in any molecular structure, in particular in a linear-chain, branched-chain or cyclic structure.

Compound (B) may have a viscosity at 25° C. ranging from 1 to 50 000 centistokes, in particular so as to be highly miscible with compound (A).

It is advantageous for compound (B) to be added in an amount such that the molecular ratio between the total amount of hydrogen atoms bonded to silicon in compound (B) and the total amount of all the ethylenically unsaturated groups in compound (A) is within the range from 1/1 to 20/1.

Compound (B) may be chosen from trimethylsiloxy-terminated methylhydropolysiloxanes, trimethylsiloxy-terminated dimethylsiloxane/methylhydrosiloxane copolymers and dimethylsiloxane/methylhydrosiloxane cyclic copolymers.

Compound (C) is the catalyst of the crosslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

The catalyst (C) is preferably added from 0.1 to 1000 parts by weight, better still from 1 to 100 parts by weight, as platinum metal proper, per 1000 parts by weight of the total amount of compounds (A) and (B).

Other organic groups may be bonded to silicon in the organopolysiloxanes (A) and (B) described previously, for instance alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The non-emulsifying silicone elastomer is generally mixed with at least one hydrocarbon-based oil and/or one silicone oil in order to form a gel. In these gels, the non-emulsifying elastomer is in the form of non-spherical particles.

The non-emulsifying organopolysiloxane elastomers used in the composition of the invention may for example be those sold under the names KSG 6 by the company Shin-Etsu; Trefil E-505C or Trefil E-506C by the company Dow Corning; Gransil (SR-CYC, SR DMF10, SR-DC556) by the company Grant Industries, or those sold in the form of gels that are already constituted: KSG 15, KSG 16, KSG 17, KSG 18, KSG 26A, KSG 26B, KSG-31, KSG-32, KSG-33, KSG-41, KSG-42, KSG-43 and KSG-44 from the company Shin-Etsu; Gransil SR 5CYC gel, Gransil SR DMF 10 gel and Gransil SR DC556 gel from the company, Gransil RPS from Grant Industries; 1229-02-167 and 1229-02-168 from the company General Electric.

Use may also be made of the silicone elastomers bearing the INCI name dimethicone/vinyl dimethicone copolymer (or polysilicone-11) as a mixture with a cyclic silicone oil. An example that may be mentioned is the mixture of crosslinked organopolysiloxane/cyclopentasiloxane or a mixture of crosslinked organopolysiloxane/cyclohexasiloxane, for instance Gransil RPS D5 or Gransil RPS D6 from the company Grant Industries.

Mention may also be made of the elastomers sold under the references DC 9040, DC 9041, DC 9509, DC 9505 and DC 9506 by the company Dow Corning. A mixture of silicone elastomers may also be used, and in particular a mixture of these commercial products.

According to one preferred embodiment of the invention, the organopolysiloxane elastomer(s) is (are) non-emulsifying. Preferably, the non-emulsifying organopolysiloxane elastomer is a dimethicone polymer crosslinked with a C₃-C₂₀ alkyl group, such as DC 9041 mentioned above.

The organopolysiloxane elastomer(s) used according to the invention is (are) present in an amount as active material which can range, for example, from 0.3% to 10% and preferably from 0.7% to 5% by weight, of the total weight of the composition.

In particular, the organopolysiloxane elastomer(s) is (are) present in an amount as active material of greater than or equal to 0.5% by weight and better still greater than or equal to 1% by weight, of the total weight of the composition.

### Aqueous Phase

The aqueous phase of the composition in accordance with the invention comprises at least water. According to the galenical form of the composition, the amount of aqueous phase can range from 50% to 99% by weight, preferably from 70% to 99% by weight and better still from 80% to 97% by weight relative to the total weight of the composition. This amount depends on the galenical form of the composition desired. The amount of water may represent all or a portion of the aqueous phase and it is generally at least 30% by weight relative to the total weight of the composition, preferably at least 50% by weight and better still at least 60% by weight.

The aqueous phase may comprise at least one hydrophilic solvent, for instance substantially linear or branched lower monoalcohols having from 1 to 8 carbon atoms, such as ethanol, propanol, butanol, isopropanol or isobutanol; polyols, such as propylene glycol, isoprene glycol, butylene glycol, pentylene glycol, glycerol, sorbitol, polyethylene glycols and derivatives thereof; and mixtures thereof.

According to one particular embodiment of the invention, the hydrophilic solvent(s), when present, is (are) present in the composition in an amount ranging from 3% to 25% by weight, relative to the total weight of the composition, and preferably from 15% to 20% by weight.

The aqueous phase may also comprise hydrophilic gelling agents. Hydrophilic gelling agents that may be mentioned, for example, include carboxyvinyl polymers such as the Carbopol® products (carbomers) and the Pemulen products (acrylate/C₁₀-C₃₀-alkyl acrylate copolymer); polyacrylamides, for instance the crosslinked copolymers sold under the names Sepigel 305 (CTFA name: polyacrylamide/C13-14 isoparaffin/laureth 7) or Simulgel 600 (CTFA name: acrylamide/sodium acryloyldimethyltaurate copolymer/isohexadecane/polysorbate 80) by the company Seppic; cellulose-based derivatives such as hydroxyethylcellulose; polysaccharides and in particular gums such as xanthan gum; and mixtures thereof; sodium polyacrylates such as the product which is sold under the name Cosmedia SP® by the company Cognis; polymers comprising at least one monomer comprising a sulfonic group, such as the monomer of 2-acrylamido-2-methylpropanesulfonic acid, in particular the product which is sold under the name Hostacerin AMPS® by the company Clariant or those which are sold under the name Aristoflex® by the company Clariant.

The amount of hydrophilic gelling agent(s), when present, is between 0.1% and 2% by weight, relative to the total weight of the composition, and preferably between 0.5% and 1.5% by weight.

### Fatty Phase

The composition in accordance with the invention comprises a fatty phase. The proportion of the fatty phase in the composition in accordance with the invention can range, for example, from 1% to 50% by weight, preferentially from 1% to 30% by weight and better still from 3% to 20% by weight, relative to the total weight of the composition.

This indicated amount does not comprise the content of lipophilic surfactants.

For the purposes of the invention, the fatty phase includes any fatty substance which is liquid at ambient temperature and atmospheric pressure, generally oils, or which is solid at ambient temperature and atmospheric pressure, like butters or waxes.

By way of solid fatty substances, mention may be made of synthetic fatty substances and fatty substances of vegetable origin. The latter can be obtained by synthesis from starting materials of vegetable origin.

The solid fatty substance is advantageously chosen from:
lanolin and derivatives thereof,
polyol ethers chosen from pentaerythrityl ethers of a polyalkylene glycol, fatty alcohol ethers of a sugar, and mixtures thereof, the pentaerythrityl ether of polyethylene glycol comprising 5 oxyethylene units (5 OE) (CTFA name: PEG-5 Pentaerythrityl Ether), the pentaerythrityl ether of polypropylene glycol comprising 5 oxypropylene units (5 OP) (CTFA name: PPG-5 Pentaerythrityl Ether), and mixtures thereof, and more particularly the PEG-5 Pentaerythrityl Ether, PPG-5 Pentaerythrityl Ether and soya bean oil mixture, sold under the name Lanolide by the company Vevy, in which mixture the constituents are in a 46/46/8 ratio by weight: 46% PEG-5 Pentaerythrityl Ether, 46% PPG-5 Pentaerythrityl Ether and 8% soya bean oil,
polymeric or non-polymeric silicone compounds,
polymeric or non-polymeric fluoro compounds,
vinyl polymers, in particular:
   olefin homopolymers and copolymers,
   hydrogenated diene homopolymers and copolymers,
   linear or branched oligomers, homopolymers or copolymers of alkyl (meth)acrylates preferably containing a C₈-C₃₀ alkyl group,
   homopolymeric and copolymeric oligomers of vinyl esters containing C₈-C₃₀ alkyl groups,
   homopolymeric and copolymeric oligomers of vinyl ethers containing C₈-C₃₀ alkyl groups,
liposoluble polyethers resulting from the polyetherification between one or more C₂-C₁₀₀ and preferably C₂-C₅₀ diols,
esters,
and/or mixtures thereof.

The solid fatty substance may be a polymer, in particular a hydrocarbon-based polymer.

Among the liposoluble polyethers that are particularly preferred are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will in particular be made of copolymers such as the long-chain alkylene oxides arranged in blocks having an average molecular weight from 1000 to 10000, for example a polyoxyethylene/polydodecyl glycol block copolymer such as the ethers of dodecanediol (22 mol) and of polyethylene glycol (45 OE) sold under the brand name Elfacos ST9 by Akzo Nobel.

Preference is given in particular, among the esters, to:
esters of a glycerol oligomer, in particular diglycerol esters, in particular condensates of adipic acid and of glycerol, for which a portion of the hydroxyl groups of the glycerols has reacted with a mixture of fatty acids, such as stearic acid, capric acid, isostearic acid and 12-hydroxystearic acid, such as in particular those sold under the brand name Softisan 649 by the company Sasol,
arachidyl propionate, sold under the brand name Waxenol 801 by the company Alzo,
phytosterol esters,
fatty acid triglycerides and derivatives thereof,
pentaerythritol esters,
esters of a diol dimer and of a diacid dimer, where appropriate esterified on their free alcohol or acid functional group(s) by acid or alcohol radicals, in particular dimer dilinoleate esters; such esters can be chosen in particular from esters with the following INCI nomenclature: bis-behenyl/isostearyl/phytosteryl dimer dilinoleyl dimer dilinoleate (Plandool G), phytosteryl isostearyl dimer dilinoleate (Lusplan PI-DA or Lusplan PHY/IS-DA), phytosteryl/isostearyl/cetyl/stearyl/behenyl dimer dilinoleate (Plandool H or Plandool S), and mixtures thereof,
mango butter, such as the product sold under the reference Lipex 203 by the company AarhusKarlshamn,
hydrogenated soya bean oil, hydrogenated coconut oil, hydrogenated rapeseed oil or mixtures of hydrogenated vegetable oils, such as the soya bean, coconut, palm and rapeseed hydrogenated vegetable oil mixture, for example the mixture sold under the reference Akogel® by the company AarhusKarlshamn (INCI name: Hydrogenated Vegetable Oil),
shea butter, in particular that having the INCI name Butyrospermum Parkii Butter, such as that sold under the reference Sheasoft® by the company AarhusKarlshamn,
cocoa butter, in particular the product which is sold under the name CT Cocoa Butter Deodorized by the company Dutch Cocoa BV or the product which is sold under the name Beurre De Cacao NCB HD703 758 by the company Barry Callebaut,
shorea butter, in particular the product which is sold under the name Dub Shorea T by the company Stearinerie Dubois,
and mixtures thereof.

The waxes capable of being used in a composition according to the invention are chosen from waxes of animal, vegetable, mineral or synthetic origin and mixtures thereof, which are solid at ambient temperature. They can be hydrocarbon-based, fluorinated and/or silicone waxes.

Mention may in particular be made, by way of examples, of hydrocarbon-based waxes, such as natural beeswax (or bleached beeswax), synthetic beeswax, carnauba wax, rice bran wax, such as that sold under the reference NC 1720 by the company Cera Rica Noda, candelilla wax, such as that sold under the reference SP 75 G by the company Strahl & Pitsch, microcrystalline waxes, such as, for example, the microcrystalline waxes having a melting point of greater than 85° C., such as the products HI-MIC® 1070, 1080, 1090 and 3080 sold by the company Nippon Seiro, ceresins or ozokerites, such as, for example, isoparaffins having a melting point of less than 40° C., such as the product EMW-0003 sold by the company Nippon Seiro, a-olefin oligomers, such as the Performa V® 825, 103 and 260 polymers sold by the company New Phase Technologies, ethylene/propylene copolymers, such as Performalene® EP 700, polyethylene waxes (preferably with a molecular weight of between 400 and 600), Fischer-Tropsch waxes or the sunflower seed wax sold by the company Koster Keunen under the reference Sunflower Wax.

Mention may also be made of silicone waxes, such as alkyl or alkoxy dimethicones having from 16 to 45 carbon atoms, or fluorinated waxes.

The other solid fatty substances that may be present in the fatty phase are, for example, fatty acids containing from 8 to 30 carbon atoms, for instance stearic acid, lauric acid or palmitic acid; fatty alcohols containing from 8 to 30 carbon atoms, for instance stearyl alcohol or cetyl alcohol and mixtures thereof (cetearyl alcohol).

The fatty phase of the composition in accordance with the invention may also comprise at least one oil. The oil(s) present in the composition may be volatile or non-volatile.

The term “oil” is intended to mean any fatty substance that is in liquid form at ambient temperature (25° C.) and at atmospheric pressure.

The volatile or non-volatile oils can be hydrocarbon-based oils, in particular of animal or vegetable origin, synthetic oils, silicone oils, fluorinated oils or mixtures thereof.

For the purposes of the present invention, the term “silicone oil” is intended to mean an oil comprising at least one silicon atom, and in particular at least one Si-0 group.

The term “hydrocarbon-based oil” is intended to mean an oil mainly comprising hydrogen and carbon atoms and optionally oxygen, nitrogen, sulfur and/or phosphorus atoms.

### Non-Volatile Oils

For the purposes of the present invention, the term “non-volatile oil” is intended to mean an oil having a vapour pressure of less than 0.13 Pa (0.01 mmHg).

The non-volatile oils can be chosen in particular from non-volatile hydrocarbon-based oils, if appropriate fluorinated, and/or non-volatile silicone oils.

Mention may in particular be made, as non-volatile hydrocarbon-based oil suitable for use in the invention, of:
hydrocarbon-based oils of animal origin,
hydrocarbon-based oils of vegetable origin, such as phytosteryl esters, such as phytosteryl oleate, phytosteryl isostearate and lauroyl/octyldodecyl/phytosteryl glutamate, for example sold under the name Eldew PS203 by Ajinomoto, triglycerides consisting of fatty acid esters of glycerol, the fatty acids of which can have varied chain lengths from C₄ to C₂₄, it being possible for the latter to be linear or branched and saturated or unsaturated; these oils are in particular heptanoic or octanoic triglycerides, wheat germ oil, sunflower oil, grape seed oil, sesame oil, maize oil, apricot oil, castor oil, shea oil, avocado oil, olive oil, soya bean oil, sweet almond oil, palm oil, rapeseed oil, cottonseed oil, hazelnut oil, macadamia oil, jojoba oil, alfalfa oil, poppy oil, pumpkinseed oil, cucumber oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, such as those sold by the company Stearineries Dubois or those sold under the names Miglyol 810®, 812® and 818® by the company Dynamit Nobel; or the refined vegetable perhydrosqualene sold under the name Fitoderm by the company Cognis;
hydrocarbon-based oils of mineral or synthetic origin, such as, for example:
   synthetic ethers containing from 10 to 40 carbon atoms;
   linear or branched hydrocarbons of mineral or synthetic origin, such as liquid petroleum, polydecenes, hydrogenated polyisobutene, such as Parleam, squalane and mixtures thereof, in particular hydrogenated polyisobutene;
   synthetic esters, such as oils of formula R₁COOR₂ in which R₁ represents the residue of a linear or branched fatty acid comprising from 1 to 40 carbon atoms and R₂ represents a hydrocarbon-based chain, in particular a branched hydrocarbon-based chain, comprising from 1 to 40 carbon atoms, provided that R₁+R₂ is ≧10. The esters can in particular be chosen from esters, in particular fatty acid esters, such as, for example:
      cetearyl octanoate, esters of isopropyl alcohol, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, such as isostearyl lactate, octyl hydroxystearate, diisopropyl adipate, heptanoates, in particular isostearyl heptanoate, octanoates, decanoates or ricinoleates of alcohols or polyalcohols, such as propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol di(2-ethylhexanoate) and mixtures thereof, benzoates of C₁₂ to C₁₅ alcohols, hexyl laurate, neopentanoic acid esters, such as isodecyl neopentanoate, isotridecyl neopentanoate, isostearyl neopentanoate or octyldodecyl neopentanoate, isononanoic acid esters, such as isononyl isononanoate, isotridecyl isononanoate or octyl isononanoate, or hydroxylated esters, such as isostearyl lactate or diisostearyl malate,
      polyol esters and pentaerythritol esters, such as dipentaerythrityl tetra hyd roxystearate/tetra isostea rate,
      esters of dimer diols and of dimer diacids, such as Lusplan DD-DA5® and Lusplan DD-DA7®, sold by the company Nippon Fine Chemical and described in application FR 03 02809,
   fatty alcohols which are liquid at ambient temperature, comprising a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol,
   higher fatty acids, such as oleic acid, linoleic acid, linolenic acid and mixtures thereof, and
   dialkyl carbonates, it being possible for the two alkyl chains to be identical or different, such as dicaprylyl carbonate, sold under the name Cetiol CC® by Cognis,
   non-volatile silicone oils, such as, for example, non-volatile polydimethylsiloxanes (PDMSs), polydimethylsiloxanes comprising alkyl or alkoxy groups which are pendent and/or at the ends of the silicone chain, which groups each have from 2 to 24 carbon atoms, phenyl silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates, dimethicones or phenyl trimethicones with a viscosity of less than or equal to 100 cSt, and mixtures thereof;
and mixtures thereof.

### Volatile Oils

For the purposes of the present invention, the term “volatile oil” is intended to mean an oil (or non-aqueous medium) which is capable of evaporating on contact with the skin in less than one hour, at ambient temperature and at atmospheric pressure. The volatile oil is a volatile cosmetic oil which is liquid at ambient temperature, having in particular a nonzero vapour pressure at ambient temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile hydrocarbon-based oils can be chosen from hydrocarbon-based oils having from 8 to 16 carbon atoms, in particular branched C₈-C₁₆ alkanes (also known as isoparaffins), such as isododecane (also known as 2,2,4,4,6-pentamethylheptane), isodecane or isohexadecane, for example the oils sold under the Isopar® or Permethyl® trade names.

Volatile oils that may also be used include volatile silicones, for instance volatile linear or cyclic silicone oils, in particular those with a viscosity 8 centistokes (8 x 10⁻⁶ m²/s), and in particular containing from 2 to 10 silicon atoms and in particular from 2 to 7 silicon atoms, these silicones optionally comprising alkyl or alkoxy groups containing from 1 to 10 carbon atoms. Mention may in particular be made, as volatile silicone oil which can be used in the invention, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane and mixtures thereof.

Use may also be made of volatile fluorinated oils, such as nonafluoromethoxybutane or perfluoromethylcyclopentane, and mixtures thereof.

It is also possible to use a mixture of the oils mentioned above.

The various fatty substances as defined previously may be chosen in a varied manner by those skilled in the art so as to prepare a composition having the desired properties, for example in terms of consistency or texture.

According to one particular embodiment of the invention, the fatty phase of the composition comprises at least one oil. Preferably, the composition in accordance with the invention comprises at least one silicone oil, for instance those which have been described previously. In particular, preference is given to hydrophilic silicones, such as the product which is sold under the name Dow Corning 2501 Cosmetic Wax by the company Dow Corning (INCI name: Bis-PEG-18 Methyl Ether Dimethylsilane).

The fatty phase can also comprise other compounds dissolved in the oils, such as gelling agents and/or structuring agents other than the silicone elastomers described previously.

These compounds can in particular be chosen from modified clays, such as hectorite and derivatives thereof, for instance the products sold under the name Bentone; gums, such as silicone gums (dimethiconol); silicone resins, such as trifluoromethyl(C₁-C₄)alkyl dimethicone and trifluoropropyl dimethicone; semicrystalline polymers, such as the product sold under the names Intelimer IPA-1 and Intelimer IPA13-6 by the company Air Products and Chemicals; and mixtures thereof.

The composition according to the invention may be in various galenical forms conventionally used for topical applications and in particular in the form of dispersions of the lotion or serum type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or wax/aqueous phase dispersions. These compositions are prepared according to the usual methods.

According to one preferred embodiment of the invention, the composition is in the form of an O/W emulsion.

In addition, the compositions used according to the invention may be more or less fluid and may have the appearance of a gel, a white or coloured cream, an ointment, a milk, a lotion, a serum, a paste or a mousse.

According to one particular embodiment of the invention, the composition is in the form of an oil-in-water emulsion. The composition is preferably in the form of an emulsified gel.

The composition preferably has a skin-friendly pH which generally ranges from 4 to 8 and preferably from 4.5 to 6.5.

The emulsions may contain at least one additional emulsifier other than the emulsifying silicone elastomers described previously. Said emulsifiers may be chosen from amphoteric, anionic, cationic or non-ionic emulsifiers, used alone or as a mixture. The emulsifiers are appropriately chosen according to the emulsion to be obtained (W/O or O/W).

The additional emulsifiers are generally present in the composition in a proportion of less than or equal to 5% by weight, preferably less than or equal to 2% by weight and even more preferentially of less than or equal to 1% by weight, relative to the total weight of the composition.

According to one particular embodiment of the invention, the composition is free of surfactants.

For the purposes of the present invention, the expression “composition free of surfactants” is intended to mean a composition comprising less than 1% by weight of surfactants, preferably 0.5% by weight, of the total weight of the composition.

For the W/O emulsions, examples of emulsifiers that may be mentioned include dimethicone copolyols such as the mixture of cyclomethicone and of dimethicone copolyol sold under the name DC 5225 C by the company Dow Corning, and alkyl dimethicone copolyols such as the lauryl methicone copolyol sold under the name Dow Corning 5200 Formulation Aid by the company Dow Corning and the cetyl dimethicone copolyol sold under the name Abil EM 90® by the company Goldschmidt, or the polyglyceryl-4 isostearate/cetyl dimethicone copolyol/hexyl laurate mixture sold under the name Abil WE 09 by the company Goldschmidt. One or more coemulsifiers can also be added thereto. The coemulsifier can advantageously be chosen from the group consisting of polyol alkyl esters. Mention may in particular be made, as polyol alkyl esters, of glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Mention may be made, for the 0/W emulsions, for example, as emulsifiers, of non-ionic surfactants and in particular esters of polyols and of fatty acid having a saturated or unsaturated chain comprising, for example, from 8 to 24 carbon atoms and better still from 12 to 22 carbon atoms, and their oxyalkylenated derivatives, that is to say derivatives comprising oxyethylene and/or oxypropylene units, such as glyceryl esters of C₈^{-C}₂₄ fatty acid, and their oxyalkylenated derivatives; polyethylene glycol esters of C₈-C₂₄ fatty acid, and their oxyalkylenated derivatives; sorbitol esters of C₈^{-C}₂₄ fatty acid, and their oxyalkylenated derivatives; fatty alcohol ethers; sugar ethers of C₈^{-C}₂₄ fatty alcohols, and mixtures thereof.

Mention may in particular be made, as glyceryl ester of fatty acid, of glyceryl stearate (glyceryl mono-, di- and/or tristearate) (CTFA name: glyceryl stearate) or glyceryl ricinoleate, and mixtures thereof.

Mention may in particular be made, as polyethylene glycol esters of fatty acids, of polyethylene glycol stearate (polyethylene glycol mono-, di- and/or tristearate) and more especially polyethylene glycol 50 OE monostearate (CTFA name: PEG-50 stearate), polyethylene glycol 100 OE monostearate (CTFA name: PEG-100 stearate) and mixtures thereof.

Use may also be made of mixtures of these surfactants, such as, for example, the product containing glyceryl stearate and PEG-100 stearate, sold under the name Arlacel 165 by the company Uniqema, and the product containing glyceryl stearate (glyceryl mono/distearate) and potassium stearate, sold under the name Tegin by the company Goldschmidt (CTFA name: glyceryl stearate SE).

Mention may be made, as fatty alcohol ethers, for example, of polyethylene glycol ethers of fatty alcohol comprising from 8 to 30 carbon atoms and in particular from 10 to 22 carbon atoms, such as polyethylene glycol ethers of cetyl alcohol, stearyl alcohol or cetearyl alcohol (mixture of cetyl alcohol and stearyl alcohol). Mention may be made, for example, of ethers comprising from 1 to 200 and preferably from 2 to 100 oxyethylene groups, such as those with the CTFA name Ceteareth-20 or Ceteareth-30, and mixtures thereof.

Mention may be made, as examples of sugar mono- or polyalkyl esters or ethers, of methyl glucose isostearate, sold under the name Isolan-IS by the company Degussa Goldschmidt, or else sucrose distearate, sold under the name Crodesta F50 by the company Croda, and sucrose stearate, sold under the name Ryoto sugar ester S 1570 by the company Mitsubishi Kagaku Foods.

Mention may also be made of lipoamino acids and salts thereof, such as monosodium and disodium acylglutamates, for instance monosodium stearoyl glutamate, sold under the name Amisoft HS-11PF, and disodium stearoyl glutamate, sold under the name Amisoft HS-21 P, by the company Ajinomoto.

In a known manner, all the compositions of the invention may contain one or more adjuvants that are common in the cosmetics and dermatology fields; hydrophilic or lipophilic gelling agents and/or thickeners; moisturizing agents; emollients; hydrophilic or lipophilic active agents; free-radical scavengers; sequestrants; antioxidants; preservatives; acidifying or basifying agents; fragrances; film-forming agents; colorants (pigments such as iron oxides and titanium dioxide, nacres, soluble dyes), and fillers; and mixtures thereof.

The amounts of these various adjuvants are those conventionally used in the fields under consideration. In particular, the amounts of active agents vary according to the desired objective and are those conventionally used in the fields under consideration, for example from 0.1% to 20% and preferably from 0.5% to 10% by weight of the total weight of the composition.

### Active Agents

The composition of the invention may comprise one or more active agents chosen, for example, from moisturizing agents, such as protein hydrolysates and polyols, for instance glycerol, glycols, for instance polyethylene glycols; natural extracts; anti-inflammatory agents; oligomeric proanthocyanidins; vitamins such as vitamin A (retinol), vitamin E (tocopherol), vitamin B5 (panthenol), vitamin B3 (niacinamide), derivatives of these vitamins (in particular esters) and mixtures thereof; caffeine; depigmenting agents such as kojic acid, hydroquinone and caffeic acid; salicylic acid and derivatives thereof; α-hydroxy acids, such as lactic acid and glycolic acid and derivatives thereof; retinoids, such as carotenoids and vitamin A derivatives; hydrocortisone; melatonin; extracts of algae, of fungi, of plants, of yeasts, of bacteria; steroids; antibacterial active agents, such as 2,4,4′-trichloro-2′-hydroxydiphenyl ether (or triclosan) or 3,4,4′-trichlorocarbanilide (or triclocarban); tensioning agents; ascorbic acid and derivatives thereof, such as 5,6-di-O-dimethylsilyl ascorbate (sold by the company Exsymol under the reference PRO-AA), the potassium salt of dl-alpha-tocopheryl-2l-ascorbyl phosphate (sold by the company Senju

Pharmaceutical under the reference Sepivital EPC), magnesium ascorbyl phosphate, sodium ascorbyl phosphate (sold by the company Roche under the reference Stay-C 50); phloroglucinol; enzymes; and mixtures thereof.

The composition may also comprise at least one UV-screening agent which may be chosen from hydrophilic, lipophilic or insoluble organic screening agents and/or inorganic pigments. It will preferentially consist of at least one hydrophilic, lipophilic or insoluble organic UV-screening agent.

The composition of the invention may also comprise one or more fillers. Mention may, for example, be made of pigments such as titanium oxide, zinc oxide or iron oxide and organic pigments; kaolin; silica; talc; boron nitride; organic spherical powders, fibres; and mixtures thereof. Examples of organic spherical powders that may be mentioned include polyamide powders and in particular Nylon® powders such as Nylon-1 or Polyamide 12, sold under the name Orgasol by the company Atochem; polyethylene powders; Teflon®; microspheres based on acrylic copolymers, such as those made of ethylene glycol dimethacrylate/lauryl methacrylate copolymer, sold by the company Dow Corning under the name Polytrap; expanded powders such as hollow microspheres and in particular the microspheres sold under the name Expancel by the company Kemanord Plast or under the name Micropearl F 80 ED by the company Matsumoto; silicone resin microbeads such as those sold under the name Tospearl by the company Toshiba Silicone; polymethyl methacrylate microspheres, sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; powders of natural organic materials such as starch powders, in particular powders of maize starch, wheat starch or rice starch, which may or may not be crosslinked, such as the starch powders crosslinked with octenyl succinate anhydride, sold under the name Dry Flo by the company Akzo Nobel. Mention may be made, as fibres, for example, of polyamide fibres, such as in particular Nylon 6 (or Polyamide 6) (INCI name: Nylon 6) fibres, Nylon 6,6 (or Polyamide 66) (INCI name: Nylon 66) fibres, or such as poly(p-phenylene terephthamide) fibres; and mixtures thereof. These fillers may be present in amounts ranging from 0.1% to 5% by weight and preferably from 0.5% to 2% by weight relative to the total weight of the composition.

Needless to say, those skilled in the art will take care to select the optional adjuvant(s) added to the composition according to the invention such that the advantageous properties intrinsically associated with the composition in accordance with the invention are not, or are not substantially, adversely affected by the envisaged addition.

The examples that follow will allow the invention to be understood more clearly, without, however, being limiting in nature. The amounts indicated are given as % by weight of starting material, unless otherwise mentioned. The names of the compounds are shown as INCI names.

### EXAMPLES

### Comparative examples: Examples 1 and 2

The following compositions are prepared.

| | | 1 | 2 |
|---|---|---|---|
| Phase | INCI name | (invention) | (comparative) |
| A | Bis-PEG-18 Methyl Ether | 2.00 | 2.00 |
| | Dimethylsilane (Dow Corning | | |
| | 2501) | | |
| | PPG-3 Myristyl Ether | 0.10 | 0.10 |
| | (Tegosoft APM from Evonik | | |
| | Goldschmidt) | | |
| | Active agent(s) | 5.00 | 5.00 |
| | Preservative(s) | 0.70 | 0.70 |
| | Polyol(s) | 15 | 15 |
| | Sodium acrylates crosspolymer-2 | 0.05 | — |
| | (and) water (and) silica | | |
| | (Aquakeep ® 10 SH NF | | |
| | sold by the company Sumimoto | | |
| | Seika) | | |
| | Water | qs | qs |
| B | Xanthan gum | 0.1 | 0.1 |
| | Sodium polyacrylate | 0.25 | 0.25 |
| | (Cosmedia SP from Cognis) | | |
| | Ammonium polyacryloyldimethyl | 0.2 | 0.2 |
| | taurate (Hostacerin AMPS from | | |
| | Clariant) | | |
| | Carbomer | 0.5 | 0.5 |
| | (Synthalen K from 3V) | | |
| C | Neutralizing agent(s) | 0.17 | 0.17 |
| D | Vinyl Dimethicone/Methicone | 1.5 | 1.5 |
| | Silsesquioxane Crosspolymer | | |
| | (KSP 100 from Shin-Etsu) | | |
| | Aluminum Starch Octenylsuccinate | 0.25 | 0.25 |
| | (Dry Flo from Akzo Nobel) | | |
| E | Dimethicone (and) Dimethicone | 8.00 | 8.00 |
| | Crosspolymer | | |
| | (Dow Corning 9041) | | |
| F | Denatured alcohol | 5.00 | 5.00 |
| | | 100.00 | 100.00 |

### Composition Preparation Protocol

The aqueous phase A is heated to 85° C. and then the gels are swollen hot in a Rayneri mixer. After cooling, phases C, D, E and F are then incorporated at ambient temperature.

### Protocol for Evaluating the Technical Effect of the Compositions (Stability, Performance During/After Application, Texture, Etc.)

Compositions 1 and 2 are stable after 2 months at ambient temperature, 4° C., 40° C. and 45° C.

### Sensory Analysis

The sensory analysis of the compositions is evaluated tactilely and visually, by a panel of 16 experts, the skin of which is normal to mixed, according to the following protocol. The panel of experts trained in the description of care products evaluates the formulations monadically. The products are packaged in standard 15 ml transparent jars that are coded. Within the same session, the samples are presented in random order to each panellist. Each product is applied and evaluated on the hand and on one cheek (0.05 ml of product/area), these areas having been cleaned beforehand.

It is observed that, in the presence of the superabsorbent polymer, composition 1 emerges as being thicker between the fingers than comparative composition 2, which reflects the increase in the “cushion effect” desired during massaging (without having to increase the amount of silicone elastomer). It is also observed that the dragging effect is increased in the presence of superabsorbent polymer, which is also reflected by a tendency to slow down the speed of penetration.

### Example 3

The following composition is prepared:

| Phase | INCI name | 3 |
|---|---|---|
| A | Bis-PEG-18 Methyl Ether Dimethylsilane | 2.00 |
| | (Dow Corning 2501) | |
| | Active agent(s) | 5.00 |
| | Preservative(s) | 0.70 |
| | Glycol(s) | 17.00 |
| | Sodium acrylates crosspolymer-2 (and) water (and) | 0.05 |
| | silica (Aquakeep ® 10 SH NF sold by the company | |
| | Sumimoto Seika) | |
| | Demineralized water | qs |
| B | Xanthan gum | 0.1 |
| | Sodium polyacrylate | 0.25 |
| | (Cosmedia SP from Cognis) | |
| | Ammonium polyacryloyldimethyl taurate | 0.2 |
| | (Hostacerin AMPS from Clariant) | |
| | Carbomer | 0.5 |
| | (Synthalen K from 3V) | |
| C | Neutralizing agent(s) | 0.17 |
| D | Vinyl Dimethicone/Methicone Silsesquioxane | 1.5 |
| | Crosspolymer | |
| | (KSP 100 from Shin-Etsu) | |
| | Aluminum Starch Octenylsuccinate | 0.25 |
| | (Dry Flo from Akzo Nobel) | |
| E | Dimethicone (and) Dimethicone Crosspolymer | 10.00 |
| | (Dow Corning 9041) | |
| F | Denatured alcohol | 5.00 |
| | | 100.00 |

### Preparation Protocol

The aqueous phase A is heated to 85° C. and then the gels are swollen hot in a Rayneri mixer. After cooling, phases C, D, E and F are then incorporated at ambient temperature.

This cosmetic composition can be used in particular as a care or makeup product.

1. A composition for topical application in the form of an oil-in-water or water-in-oil emulsion comprising at least one superabsorbent polymer and at least one organopolysiloxane elastomer, wherein the organopolysiloxane elastomer is present in an amount ranging from 0.3 to 5% by weight relative to the total weight of the composition .

2. The composition according to claim 1, in which the superabsorbent polymer(s) is (are), once hydrated, in the form of particles having a weight-average size ranging from 10 μm to 1000 μm.

3. The composition according to claim 1, in which the superabsorbent polymer(s) has (have) a capacity for absorbing water containing 0.9% of NaCl of from 10 to 100 g/g.

4. The composition according to claim 1, in which the superabsorbent polymer(s) is (are) in the form of spherical particles.

5. The composition according to claim 1, in which the superabsorbent polymer(s) is (are) present in an active material content ranging from 0.01% to 5% by weight relative to the total weight of the composition.

6. The composition according to claim 1, in which the organopolysiloxane elastomer(s) is (are) emulsifying or non-emulsifying.

7. The composition according to claim 6, in which the organopolysiloxane elastomer(s) is (are) non-emulsifying.

8. The composition according to claim 7, in which the organopolysiloxane elastomer(s) is (are) chosen from those obtained:
via a crosslinking addition reaction of a diorganosiloxane containing at least one hydrogen bonded to silicon and of a diorganopolysiloxane containing ethylenically unsaturated groups bonded to silicon;
via a dehydrogenation crosslinking condensation reaction between a hydroxyl-terminated diorganopolysiloxane and a diorganopolysiloxane containing at least one hydrogen bonded to silicon;
via a crosslinking condensation reaction of a hydroxyl-terminated diorganopolysiloxane and of a hydrolysable organopolysilane;
via thermal crosslinking of an organopolysiloxane;
via crosslinking of an organopolysiloxane by high-energy radiation.

9. The composition according to claim 7, in which the organopolysiloxane elastomer(s) is (are) obtained via a crosslinking addition reaction (A) of a diorganopolysiloxane containing at least two hydrogen atoms each bonded to a different silicon atom, and (B) of a diorganopolysiloxane containing at least two ethylenically unsaturated groups bonded to silicon.

10. The composition according to claim 7, in which the organopolysiloxane elastomer(s) is (are) obtained via a reaction of a dimethylvinylsiloxy-terminated dimethylpolysiloxane and of a trimethylsiloxy-terminated methylhydropolysiloxane, in the presence of a platinum catalyst.

11. The composition according to claim 7, in which the organopolysiloxane elastomer is a dimethicone polymer crosslinked with a C₃-C₂₀ alkyl group.

12. The composition according to claim 1, in which the organopolysiloxane elastomer(s) is (are) present in an amount as active material ranging from 0.7% to 5% by weightₛ of the total weight of the composition.

13. The composition according to claim 1, comprising at least one oil.

14. The composition according to claim 1, which is in the form of an oil-in-water emulsion.

15. A process for the cosmetic treatment of a keratin material, in which a cosmetic composition as defined in claim 1 is applied to the keratin material.

16. Use of a composition as defined in claim 1, in the cosmetics or dermatology field.

17. The composition according to claim 1, in which the superabsorbent polymer(s) has (have) a capacity for absorbing water containing 0.9% of NaCl of from 20 to 80 g/g.

18. The composition according to claim 1, in which the superabsorbent polymer(s) has (have) a capacity for absorbing water containing 0.9% of NaCl of from 30 to 80 g/g.

19. The composition according to claim 1, in which the superabsorbent polymer(s) is (are) present in an active material content ranging from 0.01% to 1% by weight relative to the total weight of the composition.

20. The composition according to claim 1, in which the superabsorbent polymer(s) is (are) present in an active material content ranging from 0.02% to 0.7% by weight relative to the total weight of the composition.
